(19) United States
(12) Patent Application Publication
(10) Pub. No.: US 2021/0110932 A1
(43) Pub. Date: Apr. 15, 2021

(54) Methods and Systems to Predict Macular Edema in a Patient's Eye Following Cataract Surgery

(71) Applicant: THE REGENTS OF THE UNIVERSITY OF MICHIGAN, Ann Arbor, MI (US)

(72) Inventors: Joshua D. Stein, Ann Arbor, MI (US); Moshiur Rahman, Ann Arbor, MI (US); Chris Andrews, Ann Arbor, MI (US)

(21) Appl. No.: 17/031,008

(22) Filed: Sep. 24, 2020

Related U.S. Application Data
(60) Provisional application No. 62/912,737, filed on Oct. 9, 2019.

(51) Int. Cl. G16H 50/30; G16H 50/20; G16H 10/60; G06N 20/00

(52) U.S. Cl. CPC G16H 50/30; G06N 20/00; G16H 10/60; G16H 50/20

(57) ABSTRACT

Example methods and systems to predict macular edema in a patient's eye following cataract surgery are disclosed. An example method includes receiving a request to determine a likelihood of macular edema occurring in a patient's eye following a cataract surgery, forming an input vector based on medical records for the patient, processing, with a machine-learning based predictor, the input vector to determine the likelihood of the macular edema occurring in the patient's eye following the cataract surgery; and providing the likelihood of the macular edema occurring in the patient's eye following the cataract surgery to a medical professional for the patient.

### RELATED APPLICATION

This patent claims the priority benefit of U.S. Provisional Patent Application Ser. No. 62/912,737, which was filed on Oct. 9, 2019. U.S. Provisional Patent Application Ser. No. 62/912,737 is hereby incorporated by reference in its entirety.

### FIELD OF THE DISCLOSURE

This disclosure relates generally to cataract surgery, and, more particularly, to methods and systems to predict macular edema in the eye of a patient following cataract surgery.

### BACKGROUND

By the year 2020, more than 30 million Americans will have cataracts in 1 or both eyes. Cataract surgery is the most common surgery in the United States. While the majority of patients undergoing cataract surgery experience excellent outcomes, a small subset of patients develop complications that can limit vision. For example, cystoid macular edema (CME) is a common complication following cataract surgery with estimates of clinically significant CME following small incision phacoemulsification ranging from 0.1% to 3.8%. Evidence of CME detectable on optical coherence tomography is even higher, ranging from 5% to 11% of cases. While there are effective medical and surgical interventions to treat postoperative CME, these treatments are not without their own adverse effects and can be costly. In one study, costs were nearly 60% higher for Medicare beneficiaries who developed CME following cataract surgery compared to others without CME.

### BRIEF DESCRIPTION OF THE DRAWINGS

FIG. 1 is a block diagram of an example macular edema predictor to determine a likelihood of macular edema occurring in a patient following a cataract surgery, in accordance with aspects of this disclosure, and shown in an example environment of use.

FIG. 2 is a block diagram of an example input vector for the machine-learning based predictor of FIG. 1.

FIG. 3 is an example user interface to present prediction results.

FIG. 4 is a flowchart representative of an example method, hardware logic and instructions for implementing the macular edema predictor of FIG. 1.

FIG. 5 is a block diagram of an example training module to train the machine-learning based predictor of FIG. 1, in accordance with aspects of this disclosure.

FIG. 6 is a flowchart representative of an example method, hardware logic and instructions for implementing the training module of FIG. 5.

FIG. 7 is a block diagram of an example computing system to implement the various user interfaces, methods, functions, etc., to determine a likelihood of macular edema occurring in a patient following a cataract surgery, in accordance with aspects of this disclosure.

The figures depict embodiments of this disclosure for purposes of illustration only. One skilled in the art will readily recognize from the following discussion that alternate embodiments of the structures and methods illustrated herein may be employed without departing from the principles set forth herein.

In general, the same reference numbers will be used throughout the drawing(s) and accompanying written description to refer to the same or like parts. The figures are not to scale. Connecting lines or connectors shown in the various figures presented are intended to represent example functional relationships and/or physical or logical couplings between the various elements.

### DETAILED DESCRIPTION

To reduce complications due to CME following cataract surgery (e.g., within 90 days following surgery), machine-learning based methods and systems to predict postoperative macular edema following cataract surgery are disclosed herein. Disclosed examples process input or feature vectors formed of data collected from a patient's medical records and processed by a machine-learning based predictor. In disclosed examples, the machine-learning based predictor is trained using medical records (structured and unstructured (free text) data found in clinical examination notes and operative reports) for previously completed cataract surgeries having known CME outcomes. Examples disclosed herein can also be used to identify risk factors associated with development of CME following cataract surgery.

While examples disclosed herein relate to predicting postoperative macular edema following cataract surgery, aspects of this disclosure can be used to predict macular edema for other types of ocular surgery such as glaucoma surgery, corneal surgery, retinal surgery, etc. Further, while examples disclosed herein relate to predicting postoperative macular edema following cataract surgery, aspects of this disclosure can be used to predict other types of complications (e.g., postoperative infection, need for additional surgery, damage to structures in the eye during surgery, etc.) arising from cataract surgery and other ocular surgeries. Further still, aspects of this disclosure can be used to predict different types of macular edema resulting from ocular surgeries including, but not limited to, CME, diabetic macular edema (DME).

As used herein, medical record refers to any number and/or type(s) medical information for a patient stored on any number and/or type(s) of medium. The medical information may be formed by, for example, a medical professional (e.g., a doctor, a nurse practitioner, a nurse, a technician, a researcher, etc.), representatives thereof, data generated by any number and/or type(s) of medical testing device(s), etc. For example, the power of the intraocular lens (IOL) measured by an ocular diagnostic test device.

Experiments have shown that aspects of this disclosure can provide a more than 6% improvement in prediction accuracy, or an accuracy rate of 97% for one database of ophthalmological medical records. Accordingly, aspects of this disclosure can be used to provide significant drops in the rates of postoperative macular edema, reduce damage that can result from postoperative macular edema by facilitating prophylactic treatment of macular edema, reduce unnecessary treatment and associated costs for unnecessarily treating patients who are at low risk of this condition, etc.

For clarity of explanation, the examples disclosed herein will focus on macular edema and cataract surgery, however, aspects of this disclosure could be used to determine the likelihood of other medical complications following other medical procedures.

Reference will now be made in detail to non-limiting examples, some of which are illustrated in the accompanying drawings.

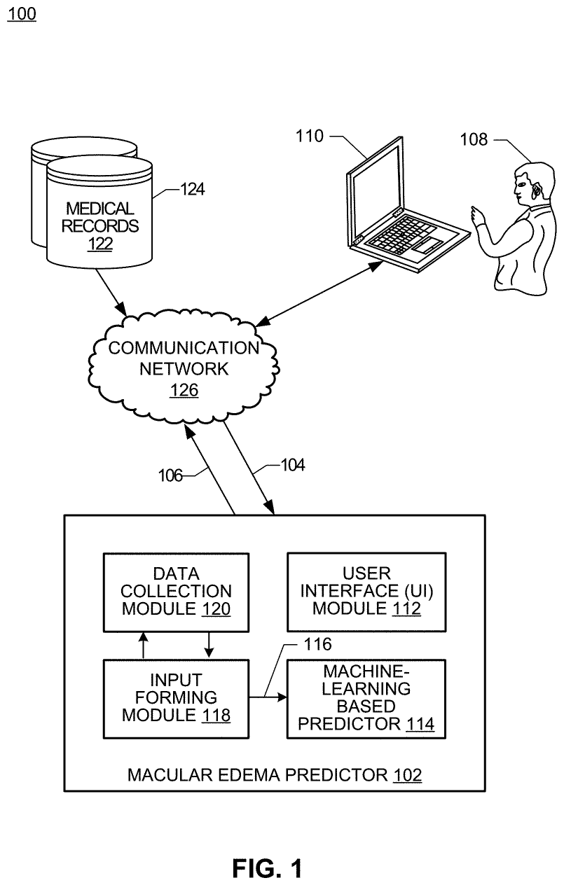

FIG. 1 is a diagram of an example system 100 that includes an example macular edema predictor 102 to, among possibly other things, determine a likelihood (e.g., a value between 0 and 1, a probability between 0% and 100%, a prediction, etc.) of macular edema in a patient's eye following cataract surgery (e.g., within 90 days post-op). In response to a request 104 regarding a patient, the macular edema predictor 102 determines a likelihood 106 of macular edema in the eye of a patient due to cataract surgery. The likelihood 106 can be determined prior to a planned, considered or completed cataract surgery. If, for example, a patient is at higher risk of macular edema, then proactive mitigating steps can be taken, such as selection of a particular surgeon with more expertise, prophylactic treatment for macular edema, justification of treatment to an insurance provider, etc. The likelihood 106 may additionally and/or alternatively be determined after surgery when determining post-surgical care. Such a likelihood may reflect, for example, that the length of surgery changed or a complication arose. In the illustrated example, the request 104 is received from a medical professional, one of which is designated at reference numeral 108, via any number or type(s) of user devices (e.g., a facsimile, a laptop computer, a tablet, a smartphone, etc.), one of which is designated at reference numeral 110. The macular edema predictor 102 provides the determined likelihood 106 for the indicated patient for presentation (e.g., display, tabulation, etc.) at the user device 110.

To receive the request 104 and provide the likelihood 106, the example macular edema predictor 102 includes any number and/or type(s) of user interface (UI) modules, one of which is designated at reference numeral 112. Example UIs 112 include a web browser interface, an application programming interface (API) for an electronic health record (EHR) client (e.g., the user device 110) interface, etc. to request and obtain a likelihood (e.g., a probability of, a prediction, etc.) of postoperative macular edema in a patient's eye, etc.

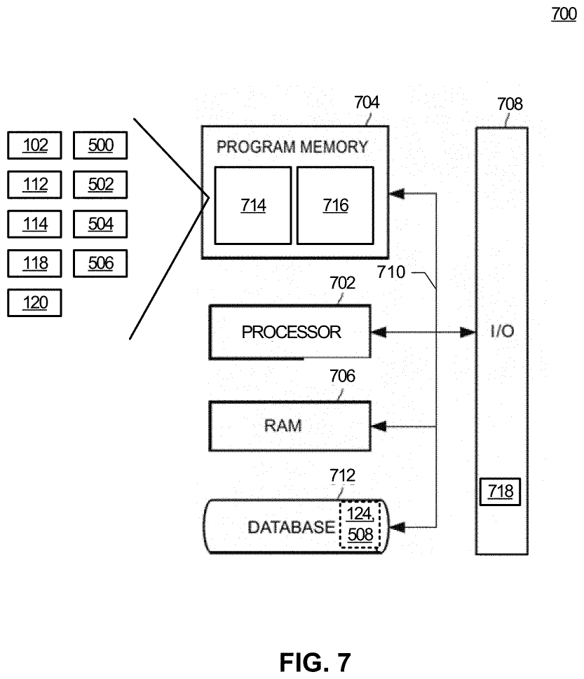

To determine a likelihood of postoperative macular edema in a patient's eye following cataract surgery, the example macular edema predictor 102 includes an example machine-learning based predictor 114. The machine-learning based predictor 114 may be, or may include a portion of a memory unit (e.g., the program memory 704 of FIG. 7) configured to store software, and machine- or computer-readable instructions that, when executed by a processing unit (e.g., the processor 702 of FIG. 7), cause the machine-learning based predictor 114 to execute a machine-learning model to determine a likelihood (e.g., a probability of, a prediction, etc.) of macular edema in a patient's eye following cataract surgery. In some examples, the machine-learning based predictor 114 implements a random forest classifier (RFC) machine-learning model that generated thousands of classification trees (each tree obtaining an optimal prediction of the CME outcome based on a small subset of the predictors) and combine them to create an overall prediction model. In some examples, a meta-classifier forms weighted averages (‘blended’) the predictions of eight other classifiers to try to boost model performance, wherein weights are derived from a regularized linear regression (LR) called Elastic Net and, thus, this model is an Elastic Net Blender (E-NETB) machine-learning model. However, any number and/or type(s) of other applicable machine learning models and/or algorithms may be used.

Additionally and/or alternatively, the machine-learning based predictor 114 could be used to determine which features of the input vector 116 for a patient were the primary contributors to the likelihood 106 for that patient. These features could be identified by comparing the likelihood 106 based on a patient's observed input vector 116 to the likelihood 106 based on other hypothetical values obtained by altering or perturbing features or input values one at a time.

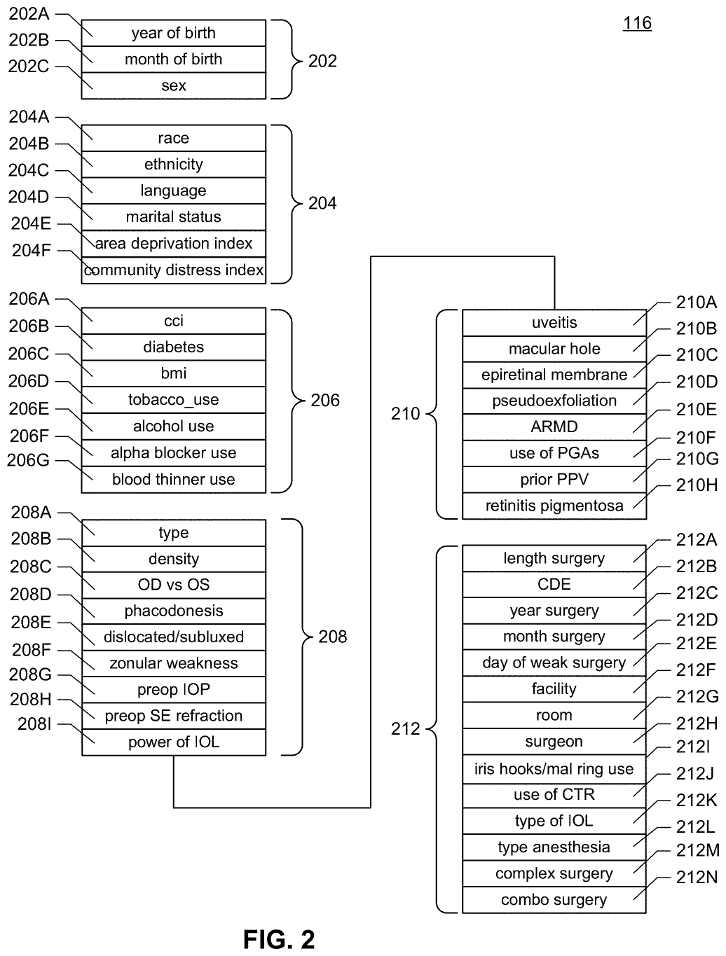

An input vector 116 including, for example, demographics 202 (see FIG. 2), social determinants of health 204, medical comorbidities 206, ocular characteristics 208, ocular comorbidities 210 and surgical details 212 is input to the machine-learning based predictor 114. In some examples, the input vector 116 is formed by an example input forming module 118. Additionally and/or alternatively, the machine-learning based predictor 114 forms the input vector 116 from the prescription 104 and the rejected claim 122. In the illustrated example of FIG. 2, the demographics 202 include a year of birth 202A, a month of birth 202B and a sex 20C; the social determinants 204 include a race 204A, an ethnicity 204B, a language 204C, a marital status 204D, an area deprivation index 204E, and a community distress index 204F; the medical comorbidities 208 include a Charlson comorbidity index (CCI) 206A, diabetes 206B, body mass index (BMI) 206C, tobacco use 206D, alcohol use 206E, alpha blocker use 206F, and blood thinner use 206G; the ocular characteristics 208 include cataract type 208A, cataract density 208B, oculus dexter (OD) vs. ocular sinister (OS) 208C, phacodonesis 208D, dislocated/subluxed 208E, zonular weakness 208F, preop interocular pressure (10P) 208G, preop spherical refractive error (SE) refraction 208H and power of the IOL 208H; the ocular comorbidities 210 include concomitant uveitis 210A, macular hole 210B, epiretinal membrane 210C, pseudoexfoliation 210D, age-related macular degeneration (ARMD) 210E, use of prostaglandin analogues (PGAs) 210F, prior pars plana vitrectomy (PPV) 210G and retinitis pigmentosa 210H; and the surgical details include length of surgery 212A, cumulative dissipative energy (CDE) 212B, year of surgery 212C, month of surgery 212D, day of week of surgery 212E, surgical facility 212F, room of surgery 212G, surgeon 212H, iris hooks/malugian ring use 212I, use of capsular tension rings (CTR) 212J, type of IOL 212K, type of anesthesia 212L, “complex” surgery 212M and combo surgery 212N.

Data and/or information can be extracted from unstructured data captured in clinical encounters and operative reports using natural language processing (NLP) to search for terms of interest. Example search algorithms considered the text immediately before and/or after one of these words or abbreviations of interest. If evidence of negation terms (e.g., “no,” “none,” “without,” etc.) existed or precautionary language such as “discussed risk of CME” were identified, the associated data was not considered evidence of the condition or complication of interest. Regular expressions and generalized Levenshtein edit distances can be used to identify close misspellings of the key terms of interest.

While an example input vector 116 is shown in FIG. 2, one or more of the fields illustrated in FIG. 2 may be combined, divided, re-arranged, omitted, eliminated or implemented in any other way. Moreover, the input vector 116 may include one or more fields in addition to or instead of those illustrated in FIG. 2. Accordingly, an input vector 116 may have more or fewer fields than that shown in FIG. 2. For example, it has been advantageously found that fewer fields (e.g., 28) can be used to predict macular edema with 95% accumulated feature impact and substantially equivalent accuracy, but with greater speed. Additionally and/or alternatively, the machine-learning based predictor 114 can learn during training which are the fields that contribute more to the determination of the likelihood 106. However, the machine-learning based predictor 114 will train faster with fewer inputs. Further, the input vector 116 may be restricted to variables that can be readily obtained.

An example input vector 116 of 28 inputs includes In the illustrated example of FIG. 2, the demographics 202 include sex 20C, race 204A, day of the week of birth, month of birth 202B, marital status 204D, tobacco use 206D, alcohol use 206E, diabetes 206B, blood thinner use 206G, surgical facility 212F, room of surgery 212G, surgeon 212H, year of surgery 212C, month of surgery 212D, day of week of surgery 212E, age at surgery, an area deprivation index 204E, and a community distress index 204F; CCI 206A, power of IOL 208H, preop 10P 208G, preop SE refraction 208H, CDE 212B, length of surgery 212A, cataract density 208B, density NS cataract, BMI 206C, and density CC cataract.

While an example input vector 116 is shown in FIG. 2, the fields shown in FIG. 2 may be combined, divided, re-arranged, omitted, eliminated or implemented in any other way. Further, the input vector 116 may include one or more fields, entries, parameters, values in addition to, or instead of those illustrated in FIG. 2, or may include more than one of any or all of the illustrated fields, entries, parameters and values. For instance, the example input vector 116 is applicable to cataract surgery and macular edema arising therefrom. When aspects of this disclosure are used to predict other medical outcomes for other medical procedures the input vector needs to have corresponding, appropriate fields.

The input forming module 118 forms the input vector 116 based on data, information, etc. that is collected, extracted, etc. from medical records 122 associated with the patient identified in the request 104. In some examples, the macular edema predictor 102 includes an example data collection module 120 to access an API of the medical record(s) 122 for the identified patient from one or more medical records database(s) 124. The medical records(s) 122 and/or medical records database(s) 124 may be associated with the same or different medical providers, medical facilities, etc. In some instances, the medical record(s) 122 may be stored in a collaborative data repository such as the Sight OUtcomes Research Collaborative (SOURCE) Ophthalmology EHR Data Repository, which stores medical records contributed by a consortium of academic ophthalmology departments. The medical records database(s) 124 may be stored on any number and/or type(s) of non-transitory computer- or machine-readable storage medium or disk.

The macular edema predictor 102, the user device 110 and the medical records database(s) 124 may be communicatively coupled via any number or type(s) of communication network(s) 126. The communication network(s) include, but are not limited to, the Internet, a local area network (LAN), a metropolitan area network (MAN), a wide area network (WAN), a wired network, a Wi-Fi® network, a cellular network, a wireless network, a satellite network, a private network, a virtual private network (VPN), etc. In some instances, secure communications are used by the data collection module 120 to obtain the medical record(s) 122.

While the example macular edema predictor 102 and/or, more generally, the example system 100 to determine a likelihood of macular edema occurring in a patient following a cataract surgery are illustrated in FIG. 1, one or more of the elements, processes and devices illustrated in FIG. 1 may be combined, divided, re-arranged, omitted, eliminated or implemented in any other way. The UI module 112, the machine-learning based predictor 114, the input forming module 118, the data collection module 120 and/or, more generally, the macular edema predictor 102 may be implemented by hardware, software, firmware and/or any combination of hardware, software and/or firmware. Thus, for example, any of the UI module 112, the machine-learning based predictor 114, the input forming module 118, the data collection module 120 and/or, more generally, the macular edema predictor 102 could be implemented by one or more of an analog or digital circuit, a logic circuit, a programmable processor, a programmable controller, a graphics processing unit (GPU), a digital signal processor (DSP), an application specific integrated circuit (ASIC), a programmable logic device (PLD), a field programmable gate array (FPGA), a field programmable logic device (FPLD), etc. Moreover, the macular edema predictor 102 and/or, more generally, the system 100 may include one or more elements, processes or devices in addition to or instead of those illustrated in FIG. 1, or may include more than one of any or all of the illustrated elements, processes and devices. For example, while not shown for clarity of illustration, the macular edema predictor 102 of FIG. 1 may include various hardware components (e.g., a processor such as the processor 702 of FIG. 7, a server, a workstation, a distributed computing system, a GPU, a DSP, etc.) that may execute software, and machine- or computer-readable instructions to estimate costs of prescriptions. For instance, the macular edema predictor 102 may interface with an EHR system, or is part of an EHR system. The macular edema predictor 102 also includes data communication components for communicating between devices.

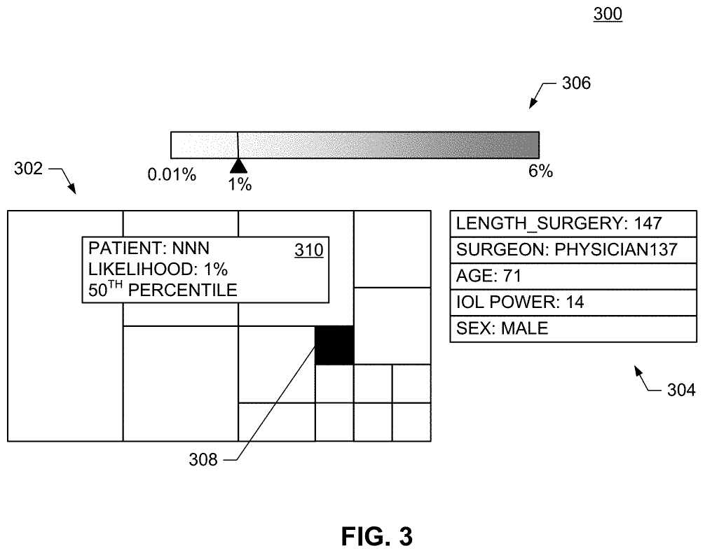

FIG. 3 is an example UI 300 in the form of a dashboard that can be presented by the UI module 112 on the user device 110 to present prediction results. The UI 300 may be used by a medical professional (e.g., a doctor, a nurse practitioner, a nurse, a researcher, etc.) to determine a likelihood 106 of postoperative macular edema in the eye of a patient prior to a planned, considered or completed cataract surgery. If, for example, a patient is at higher risk of postoperative macular edema, then mitigating steps can be taken, such as selection of a particular surgeon, prophylactic treatment for macular edema, justification of treatment to an insurance provider, etc. The likelihood 106 may additionally and/or alternatively be determined after surgery when determining post-surgical care. Such a likelihood may reflect that the length of surgery changed, a complication arose, etc.

The example user interface 300 includes a treemap 302, a metrics block 304 and a slider graph 306. The treemap 302 includes a plurality of blocks, one of which is designated at reference numeral 308, for respective ones of a plurality of patients. The size of a block 308 corresponds to the likelihood that the patient associated with the block 308 will have postoperative macular edema following cataract surgery. The larger the block, the higher the likelihood of postoperative macular edema. The blocks are nested or arranged so the patients with smaller likelihoods are generally grouped together away from patients with larger likelihoods.

When, in the illustrated example, a block (e.g., the block 308) is selected, an overlay 310 is presented. The overlay 310 of FIG. 3 identifies the patient (e.g., patient NNN), their likelihood of macular edema (e.g., 1%), and how they rank relative to other patients (e.g., in the 60^{th} percentile). When a block (e.g., the block 308) is selected, the slider graph 306 depicts the likelihood (e.g., 1%) relative to the range of likelihoods (e.g., 0.01% to 7%) for the patients represented by the blocks 308. When a block (e.g., the block 308) is selected, the metrics block 304 lists the metrics (e.g., how long surgery lasted, physician, patient's age, power of implanted lens, sex, etc.) that were the primary contributors to the patient's likelihood.

While an example UI 300 is shown in FIG. 3, one or more of the elements, graphs, blocks, data, etc. illustrated in FIG. 3 may be combined, divided, re-arranged, omitted, eliminated or implemented in any other way. Moreover, the UI 300 may include one or more elements, graphs, blocks, data, etc. in addition to or instead of those illustrated in FIG. 3, or may include more than one of any or all of the illustrated elements, graphs, blocks, data, etc. Further, prediction results may be presented using other mediums and/or having other forms. For example, a generated report may be electronically stored, transferred, retrieved and/or printed. An example report is similar to the example UI 300. However, reports may have any number and/or type(s) of elements, graphs, blocks, data, etc. arranged in any number and/or type(s) of ways.

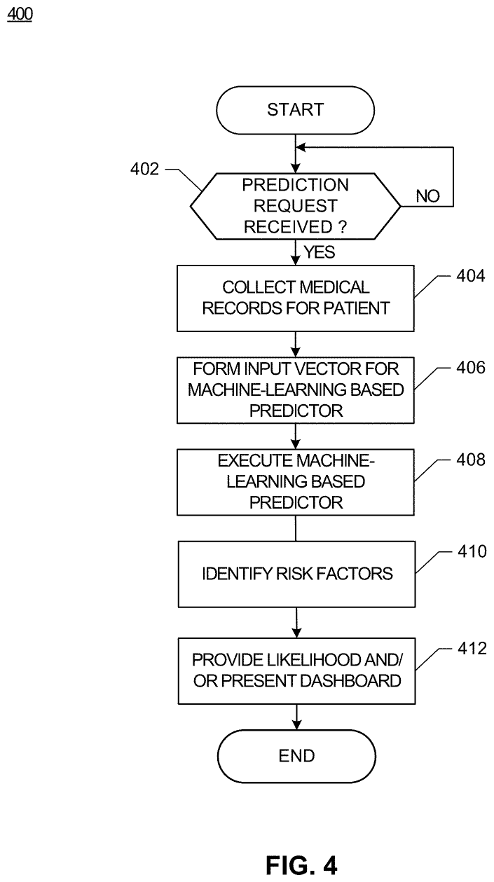

A flowchart 400 representative of example processes, methods, software, computer- or machine-readable instructions, etc. for implementing the macular edema predictor 102 is shown in FIG. 4. The processes, methods, software and instructions may be an executable program or portion of an executable program for execution by a processor such as the processor 702 of FIG. 7. The program may be embodied in software or instructions stored on any number and/or type(s) non-transitory computer- or machine-readable storage medium or disks associated with the processor 702 in which information is stored for any duration (e.g., for extended time periods, permanently, for brief instances, for temporarily buffering, and/or for caching of the information). Further, although the example program is described with reference to the flowchart illustrated in FIG. 4, many other methods of implementing the macular edema predictor 102 may alternatively be used. For example, the order of execution of the blocks may be changed, and/or some of the blocks described may be changed, eliminated, or combined. Additionally, or alternatively, any or all of the blocks may be implemented by one or more of a hardware circuit (e.g., discrete and/or integrated analog and/or digital circuitry), an ASIC, a PLD, an FPGA, an FPLD, etc. structured to perform the corresponding operation without executing software or instructions.

The example process of FIG. 4 begins with the UI module 112 waiting to receive a request to determine a likelihood of macular edema due to cataract surgery for a patient (block 402). The data collection module 120 collects one or more medical records 122 for the patient from a database 124 of medical records (block 404), and the input forming module 118 forms an input vector 116 based on the collected medical records 122 (block 406). The machine-learning based predictor 114 processes the input vector 116 to determine the requested likelihood for the patient (block 408). In some examples, the machine-learning based predictor 114 determines which features and/or values in the input vector 116 for the patient were the primary contributors to the patient's likelihood (block 410). The likelihood and/or the contributors are presented by the UI module 112 in, for example, the form of a dashboard that allows patients to be compared and contrasted (block 412).

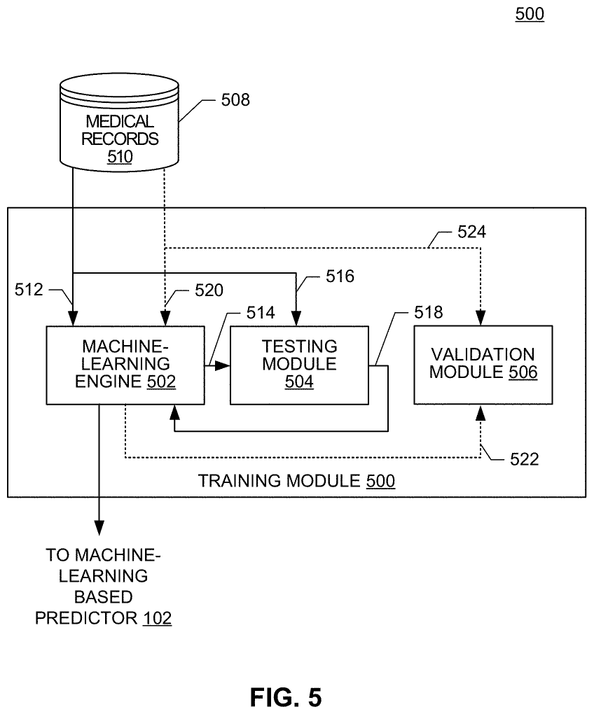

FIG. 5 is a block diagram of an example training module 500 having a machine-learning engine 502, a testing module 504 and a validation module 504. The machine-learning engine 502 can be executed for use as the machine-learning based predictor 114 of FIG. 1. The training module 500, the training module 504 and the validation module 506 may be, or may include a portion of a memory unit (e.g., the program memory 704 of FIG. 7) configured to store software, and machine- or computer-readable instructions that, when executed by a processing unit (e.g., the processor 702 of FIG. 7), cause the training module 500 to train, test and validate the machine-learning engine 502. The training module 500 includes a database 508 of training data that stores a plurality of medical records 510 for a plurality of patients on any number or type(s) of non-transitory computer- or machine-readable storage medium or disk using any number or type(s) of data structures.

Input vectors 512, as described above in connection with FIGS. 1 and 2, are formed from a portion of the medical records 510 and processed by the machine-learning engine 502 to form trial likelihoods 514. The testing module 504 compares the trial likelihoods 514 determined by the machine-learning engine 502 with actual surgical and macular edema outcomes 516 corresponding to the medical records 512 to form errors 518 that are used to develop and update the machine-learning engine 502. The machine-learning engine 502 develops, deploys and updates the machine-learning engine 502 using, for example, a random forest classifier (RFC) machine-learning model, an elastic net blender (E-NETB) machine-learning model, etc.

To validate the developing machine-learning engine 506, the training module 500 includes the validation module 506. The validation module 506 statistically validates the developing machine-learning engine 502 using, for example, k-fold cross-validation. The medical records 510 are randomly split into k parts (e.g., 5 parts). The developing machine-learning engine 502 is trained using k−1 parts 512 of the k parts of the medical records 510 to form the trial likelihoods 514. The machine-learning engine 502 is evaluated using the remaining 1 (one) part 520 of the medical records 510 to which the machine-learning engine 502 has not been exposed. Outputs 522 of the developing machine-learning engine 502 for the medical records 520 are compared to actual surgical and macular edema outcomes 524 for the medical records 510 by the validation module 506 to determine the performance or convergence of developing machine-learning engine 502. Performance or convergence can be determined by, for example, identifying when a metric computer over the errors (e.g., a mean-square metric, a rate-of-decrease metric, etc.) satisfies a criteria (e.g., a metric is less than a predetermined threshold, such as a root mean squared error). In some examples, each of the k parts includes 16% of the medical records 510, with 20% of the medical records 510 reserved.

While the machine-learning engine 502, the testing module 504, the validation module 506 and/or, more generally, the training module 500 are illustrated in FIG. 5, one or more of the elements, processes and devices illustrated in FIG. 5 may be combined, divided, re-arranged, omitted, eliminated or implemented in any other way. The machine-learning engine 502, the testing module 504, the validation module 506 and/or, more generally, the training module 500 may be implemented by hardware, software, firmware and/or any combination of hardware, software and/or firmware. Thus, for example, any of the machine-learning engine 502, the testing module 504, the validation module 506 and/or, more generally, the training module 500 could be implemented by one or more of an analog or digital circuit, a logic circuit, a programmable processor, a programmable controller, a GPU, a DSP, an ASIC, a PLD, an FPGA, an FPLD, etc. Moreover, the training module 500 may include one or more elements, processes or devices in addition to or instead of those illustrated in FIG. 5, or may include more than one of any or all of the illustrated elements, processes and devices. For example, while not shown for clarity of illustration, the training module 500 of FIG. 5 may include various hardware components (e.g., a processor such as the processor 702 of FIG. 7, a server, a workstation, a distributed computing system, a GPU, a DSP, etc.) that may execute software, and machine- or computer-readable instructions to estimate costs of prescriptions. The training module 500 also includes data communication components for communicating between devices.

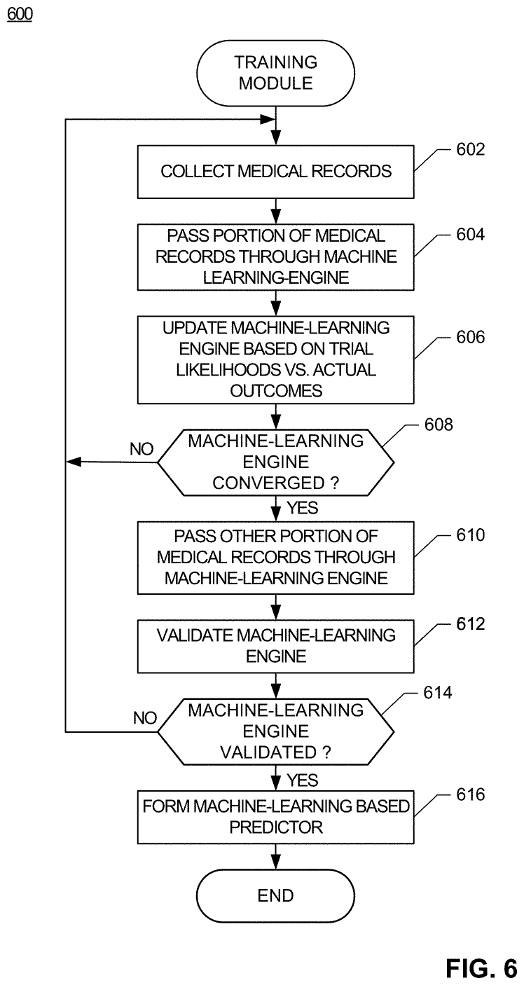

A flowchart 600 representative of example processes, methods, software, firmware, and computer- or machine-readable instructions for implementing the training module 500 is shown in FIG. 6. The processes, methods, software and instructions may be an executable program or portion of an executable program for execution by a processor such as the processor 702 of FIG. 7. The program may be embodied in software or instructions stored on a non-transitory computer- or machine-readable storage medium or disk associated with the processor 702. Further, although the example program is described with reference to the flowchart illustrated in FIG. 6, many other methods of implementing the training module 500 may alternatively be used. For example, the order of execution of the blocks may be changed, and/or some of the blocks described may be changed, eliminated, or combined. Additionally, or alternatively, any or all of the blocks may be implemented by one or more hardware circuits (e.g., discrete and/or integrated analog and/or digital circuitry, an ASIC, a PLD, an FPGA, an FPLD, a logic circuit, etc.) structured to perform the corresponding operation without executing software or firmware.

The example process of FIG. 6 begins with collecting a plurality of medical records 510 for a plurality of patients (block 602). Medical records 512 representing k−1 parts of the medical records 510 are passed through the machine-learning engine 502 (block 604), and the machine-learning engine 502 is updated based on comparisons by the testing module 504 of the outputs 514 of the machine-learning engine 502 (block 606). If training of the machine-learning engine 502 has not converged (block 608), control returns to block 604 to continue training the machine-learning engine 502. If training of the machine-learning engine 502 has converged (block 608), the medical claims 520 of the remaining portion of the medical records 510 are passed through the machine-learning engine 502 (block 610), and outputs 522 of the machine-learning engine 502 are used by the validation module 506 to validate the machine-learning engine 502 (block 612). If the machine-learning engine 502 validates (block 614), the machine-learning engine 502 is used to form the machine-learning based predictor 114 (block 616) (e.g., coefficients are copied, etc.), and control exits from the example process of FIG. 6. Otherwise, if the machine-learning engine 502 does not validate (block 614), then control returns to block 602 to continue training.

As mentioned above, the example processes of FIGS. 4 and 6 may be implemented using executable instructions (e.g., computer and/or machine-readable instructions) stored on a non-transitory computer and/or machine-readable medium such as a hard disk drive, a flash memory, a read-only memory, a CD, a CD-ROM, a DVD, a cache, a random-access memory and/or any other storage device or storage disk in which information is stored for any duration (e.g., for extended time periods, permanently, for brief instances, for temporarily buffering, and/or for caching of the information). As used herein, the term non-transitory computer-readable medium is expressly defined to include any type of computer-readable storage device and/or storage disk and to exclude propagating signals and to exclude transmission media.

Referring now to FIG. 7, a block diagram of an example computing system 700 that may be used to, for example, implement all or part of the macular edema predictor 102 of FIG. 1 and/or the training module 500 of FIG. 5 is shown. The computing system 700 may be, for example, a server, a personal computer, a workstation, a self-learning machine (e.g., a neural network), a mobile device (e.g., a cell phone, a smart phone, a tablet such as an IPAD™), a personal digital assistant (PDA), an Internet appliance, a DVD player, a CD player, a digital video recorder, a Blu-ray player, a gaming console, a personal video recorder, a set top box, a headset or other wearable device, or any other type of computing device

The computing system 700 includes a processor 702, a program memory 704, a RAM 706, and an input/output (I/O) circuit 708, all of which are interconnected via an address/data bus 710. The program memory 704 may store software, and machine- or computer-readable instructions (e.g., representing some or all the macular edema predictor 102, the UI module 112, the machine-learning based predictor 114, the input forming module 118, the data collection module 120, the training module 500, the machine-learning engine 502, the testing module 504 and/or the validation module 506), which may be executed by the processor 702.

It should be appreciated that although FIG. 7 depicts only one processor 702, the computing system 700 may include multiple processors 702. Moreover, different portions of the macular edema predictor 102 and/or the training module 500 may be implement by different computing systems such as the computing system 700. The processor 702 of the illustrated example is hardware, and may be a semiconductor based (e.g., silicon based) device. Example processors 702 include a programmable processor, a programmable controller, a GPU, a DSP, an ASIC, a PLD, an FPGA, an FPLD, etc. In this example, the processor 702 implements all or part of the macular edema predictor 102, the UI module 112, the machine-learning based predictor 114, the input forming module 118, the data collection module 120, the training module 500, the machine-learning engine 502, the testing module 504 and/or the validation module 506.

The program memory 704 may include volatile and/or non-volatile memories, for example, one or more RAMs (e.g., a RAM 714) or one or more program memories (e.g., a ROM 716), or a cache (not shown) storing one or more corresponding software, and machine- or computer-instructions. For example, the program memory 704 stores software, machine- or computer-readable instructions, or machine- or computer-executable instructions that may be executed by the processor 702 to implement all or part of the macular edema predictor 102, the UI module 112, the machine-learning based predictor 114, the input forming module 118, the data collection module 120, the training module 500, the machine-learning engine 502, the testing module 504 and/or the validation module 506. Modules, systems, etc. instead of and/or in addition to those shown in FIG. 7 may be implemented. The software, machine-readable instructions, or computer-executable instructions may be stored on separate non-transitory computer- or machine-readable storage mediums or disks, or at different physical locations. Example memories 704, 714, 716 include any number or type(s) of volatile or non-volatile non-transitory computer- or machine-readable storage medium or disks.

In some embodiments, the processor 702 may also include, or otherwise be communicatively connected to, a database 712 or other volatile or non-volatile non-transitory computer- or machine-readable storage medium or disk. In the illustrated example, the database 712 stores the medical records 122 and/or 510.

Although FIG. 7 depicts the I/O circuit 708 as a single block, the I/O circuit 708 may include a number of different types of I/O circuits or components that enable the processor 702 to communicate with peripheral I/O devices. Example interface circuits 708 include an Ethernet interface, a universal serial bus (USB), a Bluetooth® interface, a near field communication (NFC) interface, and/or a PCI express interface. The peripheral I/O devices may be any desired type of I/O device such as a keyboard, a display (a liquid crystal display (LCD), a cathode ray tube (CRT) display, a light emitting diode (LED) display, an organic light emitting diode (OLED) display, an in-place switching (IPS) display, a touch screen, etc.), a navigation device (a mouse, a trackball, a capacitive touch pad, a joystick, etc.), a speaker, a microphone, a printer, a button, a communication interface, an antenna, etc.

The I/O circuit 708 may include any number of network transceivers 718 that enable the computing system 700 to communicate with other computer systems or components that implement other portions of the system 100 or the training module 500 via, e.g., a network (e.g., the Internet). The network transceiver 718 may be a wireless fidelity (Wi-Fi) transceiver, a Bluetooth transceiver, an infrared transceiver, a cellular transceiver, an Ethernet network transceiver, an asynchronous transfer mode (ATM) network transceiver, a digital subscriber line (DSL) modem, a dialup modem, a satellite transceiver, a cable modem, etc.

Example methods and systems to predict macular edema in a patient's eye following cataract surgery are disclosed herein. Further examples and combinations thereof include at least the following.

Example 1 is a method to determine a likelihood of macular edema including: receiving a request to determine a likelihood of macular edema occurring in a patient's eye following a cataract surgery; forming an input vector based on medical records for the patient; processing, with a machine-learning based predictor, the input vector to determine the likelihood of the macular edema occurring in the patient's eye following the cataract surgery; and providing the likelihood of the macular edema occurring in the patient's eye following the cataract surgery to a medical professional for the patient.

Example 2 is the method of example 1, further comprising providing risk factors associated with the likelihood.

Example 3 is the method of example 1 or example 2, further comprising providing possible mitigating factors.

Example 4 is the method of any of examples 1 to 3, further comprising providing an electronic health record system configured to: store the medical records; and provide a user interface to receive the request and provide the likelihood in response to the request.

Example 5 is the method of any of examples 1 to 4, further comprising training the machine-learning based predictor with medical records for a plurality of patients, the medical records including, for each patient, an indication of whether of macular edema occurred following a respective cataract surgery to their eye.

Example 6 is the method of example 5, further comprising: training the machine-learning based predictor with a first portion of the medical records for the plurality of patients; and validating the machine-learning based predictor with a second portion of the medical records for the plurality of patients.

Example 7 is the method of example 6, further comprising obtaining the medical records for the plurality of patients from a collaborative health records database.

Example 8 is the method of any of examples 1 to 7, wherein the input vector includes at least one of demographics, social determinants of health, medical comorbidities, surgical details, ocular characteristics, or ocular comorbidities.

Example 9 is a system including: a first interface configured to receive a request to determine a probability of postoperative macular edema following a cataract surgery; an input forming module configured to form an input vector based on medical records associated with the patient; a machine-learning based predictor configured to process the input vector to determine the probability of the postoperative macular edema following the cataract surgery; and a second interface configured to provide the probability of the postoperative macular edema following the cataract surgery to a medical professional for the patient.

Example 10 is the system of example 9, further comprising an electronic health records system including: a non-transitory computer-readable storage medium storing the medical records; the first interface; the second interface; and a third interface to the machine-learning based predictor.

Example 11 is the system of example 9 or example 10, further comprising a training module configured to train the machine-learning based predictor with medical records for a plurality of patients, the medical records including, for each patient, an indication of whether macular edema occurred following a respective cataract surgery to their eye.

Example 12 is the system of example 11, wherein the training module is further configured to: train the machine-learning based predictor with a first portion of the medical records for the plurality of patients; and validate the machine-learning based predictor with a second portion of the medical records for the plurality of patients.

Example 13 is the system of example 11, further comprising a data collection module to obtain the medical records for the plurality of patients from a collaborative health records database.

Example 14 is the system of any of examples 9 to 13, wherein the input vector includes at least one of demographics, social determinants of health, medical comorbidities, surgical details, ocular characteristics, or ocular comorbidities.

Example 15 is the system of any of examples 9 to 14, wherein the machine-learning based predictor identifies risk factors associated with the likelihood.

Example 16 is a non-transitory computer-readable storage medium comprising instructions that, when executed, cause a machine to: receive a request to determine a likelihood of swelling in an eye of a patient following a surgery to the eye; form an input vector based on medical records for the patient; process, with a machine-learning based predictor, the input vector to determine the likelihood of the swelling in the eye following the surgery to the eye; and provide the likelihood of the swelling in the eye following the surgery to the eye to a medical professional for the patient.

Example 17 is the non-transitory computer-readable storage medium of example 16, including further instructions that, when executed, cause the machine to train the machine-learning based predictor with medical records for a plurality of patients, the medical records including, for each patient, an indication of whether of macular edema occurred following a respective cataract surgery.

Example 18 is the non-transitory computer-readable storage medium of example 17, including further instructions that, when executed, cause the machine to: training the machine-learning engine with a first portion of the medical records for the plurality of patients; and validating the machine-learning engine with a second portion of the medical records for the plurality of patients.

Example 19 is the non-transitory computer-readable storage medium of any of examples 16 to 18, including further instructions that, when executed, cause the machine to obtain the medical records for the plurality of patients from a collaborative health records database.

Example 20 is the non-transitory computer-readable storage medium of any of examples 16 to 19, wherein the input vector includes at least one of demographics, social determinants of health, medical comorbidities, surgical details, ocular characteristics, or ocular comorbidities.

As used herein, a non-transitory computer- or machine-readable storage medium or disk may be, but is not limited to, one or more of a compact disc (CD), a compact disc read-only memory (CD-ROM), a hard disk drive (HDD), a solid state drive (SDD), a digital versatile disk (DVD), a Blu-ray disk, a cache, a redundant array of independent disks (RAID) system, a flash memory, a read-only memory (ROM), a random access memory (RAM), an optical storage drive, a semiconductor memory, a magnetically readable memory, an optically readable memory, a solid-state storage device, or any other storage device or storage disk in which information may be stored for any duration (e.g., permanently, for an extended time period, for a brief instance, for temporarily buffering, for caching of the information, etc.). As used herein, the term non-transitory machine-readable medium is expressly defined to exclude propagating signals and to exclude transmission media.

Use of “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise. A device or structure that is “configured” in a certain way is configured in at least that way, but may also be configured in ways that are not listed.

Further, as used herein, the expressions “in communication,” “coupled” and “connected,” including variations thereof, encompasses direct communication and/or indirect communication through one or more intermediary components, and does not require direct mechanical or physical (e.g., wired) communication and/or constant communication, but rather additionally includes selective communication at periodic intervals, scheduled intervals, aperiodic intervals, and/or one-time events. The embodiments are not limited in this context.

Further still, unless expressly stated to the contrary, “or” refers to an inclusive or and not to an exclusive or. For example, “A, B or C” refers to any combination or subset of A, B, C such as (1) A alone, (2) B alone, (3) C alone, (4) A with B, (5) A with C, (6) B with C, and (7) A with B and with C. As used herein, the phrase “at least one of A and B” is intended to refer to any combination or subset of A and B such as (1) at least one A, (2) at least one B, and (3) at least one A and at least one B. Similarly, the phrase “at least one of A or B” is intended to refer to any combination or subset of A and B such as (1) at least one A, (2) at least one B, and (3) at least one A and at least one B.

Moreover, in the foregoing specification, specific embodiments have been described. However, one of ordinary skill in the art appreciates that various modifications and changes can be made in view of aspects of this disclosure without departing from the scope of the invention as set forth in the claims below. Accordingly, the specification and figures are to be regarded in an illustrative rather than a restrictive sense, and all such modifications made in view of aspects of this disclosure are intended to be included within the scope of present aspects.

Additionally, the benefits, advantages, solutions to problems, and any element(s) that may cause any benefit, advantage, or solution to occur or become more pronounced are not to be construed as a critical, required, or essential features or elements of any or all the claims.

Furthermore, although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

Finally, any references, including, but not limited to, publications, patent applications, and patents cited herein are hereby incorporated in their entirety by reference to the same extent as if each reference were individually and specifically indicated to be incorporated by reference and were set forth in its entirety herein.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

What is claimed is:

1. A method to determine a likelihood of macular edema, the method comprising:
receiving a request to determine a likelihood of macular edema occurring in a patient's eye following a cataract surgery;
forming an input vector based on medical records for the patient;
processing, with a machine-learning based predictor, the input vector to determine the likelihood of the macular edema occurring in the patient's eye following the cataract surgery; and
providing the likelihood of the macular edema occurring in the patient's eye following the cataract surgery to a medical professional for the patient.

2. The method of claim 1, further comprising providing risk factors associated with the likelihood.

3. The method of claim 1, further comprising providing possible mitigating factors.

4. The method of claim 1, further comprising providing an electronic health record system configured to:
store the medical records; and
provide a user interface to receive the request and provide the likelihood in response to the request.

5. The method of claim 1, further comprising training the machine-learning based predictor with medical records for a plurality of patients, the medical records including, for each patient, an indication of whether of macular edema occurred following a respective cataract surgery to their eye.

6. The method of claim 5, further comprising:
training the machine-learning based predictor with a first portion of the medical records for the plurality of patients; and
validating the machine-learning based predictor with a second portion of the medical records for the plurality of patients.

7. The method of claim 6, further comprising obtaining the medical records for the plurality of patients from a collaborative health records database.

8. The method of claim 1, wherein the input vector includes at least one of demographics, social determinants of health, medical comorbidities, surgical details, ocular characteristics, or ocular comorbidities.

9. A system, comprising:
a first interface configured to receive a request to determine a probability of postoperative macular edema following a cataract surgery;
an input forming module configured to form an input vector based on medical records associated with the patient;
a machine-learning based predictor configured to process the input vector to determine the probability of the postoperative macular edema following the cataract surgery; and
a second interface configured to provide the probability of the postoperative macular edema following the cataract surgery to a medical professional for the patient.

10. The system of claim 9, further comprising an electronic health records system including:
a non-transitory computer-readable storage medium storing the medical records;
the first interface;
the second interface; and
a third interface to the machine-learning based predictor.

11. The system of claim 9, further comprising a training module configured to train the machine-learning based predictor with medical records for a plurality of patients, the medical records including, for each patient, an indication of whether macular edema occurred following a respective cataract surgery to their eye.

12. The system of claim 11, wherein the training module is further configured to:
train the machine-learning based predictor with a first portion of the medical records for the plurality of patients; and
validate the machine-learning based predictor with a second portion of the medical records for the plurality of patients.

13. The system of claim 11, further comprising a data collection module to obtain the medical records for the plurality of patients from a collaborative health records database.

14. The system of claim 9, wherein the input vector includes at least one of demographics, social determinants of health, medical comorbidities, surgical details, ocular characteristics, or ocular comorbidities.

15. The system of claim 9, wherein the machine-learning based predictor identifies risk factors associated with the likelihood.

16. A non-transitory computer-readable storage medium comprising instructions that, when executed, cause a machine to:
receive a request to determine a likelihood of swelling in an eye of a patient following a surgery to the eye;
form an input vector based on medical records for the patient;
process, with a machine-learning based predictor, the input vector to determine the likelihood of the swelling in the eye following the surgery to the eye; and
provide the likelihood of the swelling in the eye following the surgery to the eye to a medical professional for the patient.

17. The non-transitory computer-readable storage medium of claim 16, including further instructions that, when executed, cause the machine to train the machine-learning based predictor with medical records for a plurality of patients, the medical records including, for each patient, an indication of whether of macular edema occurred following a respective cataract surgery.

18. The non-transitory computer-readable storage medium of claim 17, including further instructions that, when executed, cause the machine to:
training the machine-learning engine with a first portion of the medical records for the plurality of patients; and
validating the machine-learning engine with a second portion of the medical records for the plurality of patients.

19. The non-transitory computer-readable storage medium of claim 16, including further instructions that, when executed, cause the machine to obtain the medical records for the plurality of patients from a collaborative health records database.

20. The non-transitory computer-readable storage medium of claim 16, wherein the input vector includes at least one of demographics, social determinants of health, medical comorbidities, surgical details, ocular characteristics, or ocular comorbidities.
